(19) United States
(12) Patent Application Publication
(10) Pub. No.: US 2025/0114318 A1
(43) Pub. Date: Apr. 10, 2025

(54) COMPOSITION AND USE THEREOF FOR MANUFACTURING FOOD FOR PREVENTION OF SARCOPENIA

(71) Applicant: HUI-YU HUNG, Taichung City (TW)

(72) Inventor: HUI-YU HUNG, Taichung City (TW)

(21) Appl. No.: 18/482,321

(22) Filed: Oct. 6, 2023

(51) Int. Cl. A61K 31/205; A61K 35/20; A61K 36/062; A61K 38/16; A61P 21/00

(52) U.S. Cl. CPC A61K 31/205; A61K 35/20; A61K 36/062; A61K 38/168; A61P 21/00

(57) ABSTRACT

A composition is provided, wherein the composition includes 70.00-99.00 weight percentage (wt %) of phytopeptide, 0.10-20.00 wt % of branched-chain amino acid, 0.10-20.00 wt % of whey protein concentrate, 0.10-20.00 wt % of creatine, and 0.10-20.00 wt % of chromium yeast. A use of composition as described above is further provided.

### BACKGROUND OF THE INVENTION

### Field of the Invention

The present invention relates to a composition and a use thereof for manufacturing food for prevention of sarcopenia.

### Description of the Prior Art

Sarcopenia may be caused by the aging process, chronic diseases (such as cardiovascular disease, heart disease, cancer, etc.), malnutrition, etc. It will cause the continuous reduction of muscle mass and muscle function throughout the whole body, and then the loss of muscle function. Can and affect the quality of life. The conventional technology is to supplement nutrition (such as nutrition from food, such as milk, soybeans, peanuts, chicken, etc.) with exercise training to effectively improve sarcopenia, increase muscle mass and strengthen muscle strength. However, for those who do less exercise, it is a hard thing to do exercise training combined with nutritional supplements to improve sarcopenia, which can easily lead to poor execution and ineffective results.

Glutathione serves as an antioxidant in animal and human cells, and it can be used to slow down aging, reduce the risk of cancer, etc. Therefore, when the expression level of glutathione is low, it cannot resist the entry of free radicals into the body, causing damage to tissues and cells. In addition, the ubiquitin-proteasome system (UPS) plays an important role in protein degradation during muscle atrophy. Currently, there is no supplements to regulate expressing of glutathione or/and ubiquitin-proteasome system to prevent sarcopenia.

The present invention is, therefore, arisen to obviate or at least mitigate the above-mentioned disadvantages.

### SUMMARY OF THE INVENTION

The main object of the present invention is to provide a composition and a use thereof for manufacturing food for prevention of sarcopenia.

To achieve the above and other objects, a composition is provided, wherein the composition includes 70.00-99.00 weight percentage (wt %) of phytopeptide, 0.10-20.00 wt % of branched-chain amino acid, 0.10-20.00 wt % of whey protein concentrate, 0.10-20.00 wt % of creatine, and 0.10-20.00 wt % of chromium yeast.

To achieve the above and other objects, a use of the composition is further provided.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 shows that a time course of C57BL/6 mice inoculated with Lewis lung carcinoma (LLC) cells and treated with composition A according to a preferred embodiment of the present invention;

FIG. 2 shows that the effect of composition A on the weight of C57BL/6 mice induced by Lewis lung carcinoma cells according to a preferred embodiment of the present invention;

FIG. 3 shows that the effect of composition A on the size of tumor of C57BL/6 mice induced by Lewis lung carcinoma cells according to a preferred embodiment of the present invention;

FIG. 4 shows that the effect of composition A on the weight of tumor of C57BL/6 mice induced by Lewis lung carcinoma cells according to a preferred embodiment of the present invention;

FIG. 5 shows that the effect of composition A on the gastrocnemius atrophy in C57BL/6 mice induced by Lewis lung carcinoma cells according to a preferred embodiment of the present invention;

FIG. 6 shows that the effect of composition A on the weight of the gastrocnemius muscle of C57BL/6 mice induced by Lewis lung carcinoma cells according to a preferred embodiment of the present invention;

FIG. 7 shows a gastrocnemius muscle tissue section of a mouse according to a preferred embodiment of the present invention;

FIG. 8 shows the performance of composition A in inhibiting nitric oxide according to a preferred embodiment of the present invention;

FIG. 9 shows the performance of composition A in activating glutathione according to a preferred embodiment of the present invention;

FIG. 10 shows the performance of composition A in inhibiting muscle-specific E3 ubiquitin ligase according to a preferred embodiment of the present invention; and

FIG. 11 shows the performance of composition A in inhibiting inflammatory protein in gastrocnemius muscle according to a preferred embodiment of the present invention.

### DETAILED DESCRIPTION OF THE PREFERRED EMBODIMENTS

A composition of the present invention includes 70.00-99.00 weight percentage (wt %) of phytopeptide, 0.10-20.00 wt % of branched-chain amino acid, 0.10-20.00 wt % of whey protein concentrate, 0.10-20.00 wt % of creatine, and 0.10-20.00 wt % of chromium yeast. Preferably, the composition includes 85.90-95.90 wt % of phytopeptide, 0.25-1.25 wt % of branched-chain amino acid, 1.85-5.85 wt % of whey protein concentrate, 1.00-5.00 wt % creatine and 0.50-2.00 wt % chromium yeast. Specifically, the composition includes 90.90 wt % of phytopeptide, 0.75 wt % of branched-chain amino acid, 3.85 wt % of whey protein concentrate, 3.00 wt % creatine and 1.50 wt % of chromium yeast.

In this embodiment, the phytopeptide is from 2834.70 mg to 3164.70 mg, the branched-chain amino acid is from 8.25 mg to 41.25 mg, the whey protein concentrate is from 61.40 mg to 193.05 mg, the creatine is from 33.00 mg to 165.00 mg, and the chromium yeast is from 16.50 mg to 66.00 mg. Preferably, the phytopeptide is from 2950.00 mg to 3050.00 mg, the branched-chain amino acid is from 20.00 mg to 30.00 mg, the whey protein concentrate is from 120.00 mg to 130.00 mg, the creatine is from 50.00 mg to 150.00 mg, and the chromium yeast is from 45.00 mg to 55.00 mg.

Preferably, the phytopeptide is ipomoea batatas phytopeptide.

### [Manufacturing of Ipomoea Batatas Phytopeptide]

The ipomoea batatas phytopeptide extracted and purified from ipomoea batatas (L.) Lam. which can be purchased from the market. 1. The ipomoea batatas (L.) Lam. is peeled, washed and cut into strips. 2. Four unit volumes containing 100 mM (1 mol/m3) sodium chloride (NaCl), 1 w/v % (weight/volume percentage concentration, w/v %) of ascorbic acid in salt and 1 w/v % of polyvinyl polyrrolidone (PVPP) in 100 mM Tris-HCl buffer solution (pH 7.9) are extracted in a homogenizer. 3. It is carried out to filter through four layers of cotton cloth and extract from the homogenizer, and the homogenate was then centrifuged at 12,000×g for 20-40 minutes several times. 4. Then, the crude extract was loaded into a Trypsin-Sepharose 4B affinity column (1.0 cm×10 cm), and the adsorbed ipomoea batatas phytopeptide was washed by 0.2 M (molarity) KCl buffer solution (pH 2.0). 5. Desalt the extract, concentrate it with Centricon 10, and then proceeds with freeze-dry. SDS-PAGE analysis of the purified ipomoea batatas phytopeptide showed a monomer with a molecular mass of approximately 25 kDa (kilodalton). The yield is 8.3% (the ratio of 150 mg of purified TI protein to 1,800 mg of total protein in the crude extract). The ipomoea batatas phytopeptide may finally be made into powder form.

A use of the composition for manufacturing food for prevention of sarcopenia is further provided. The food may be health food, beverage, fermented food, baked products, dietary supplements, etc. In addition, the form of the composition is powder, tablet, granulation or microcapsule.

The food containing the composition is capable of slowing down muscle atrophy, inhibiting tumor growth and cachexia induced by tumors, activating glutathione, inhibiting muscle-specific ubiquitin ligase, inhibiting inflammatory protein in gastrocnemius muscle, inhibiting nitric oxide, and/or inhibiting tumor-induced muscle atrophy.

The following experimental data are provided for specific explanation.

In this experiment, Lewis lung carcinoma (LLC) cells were used to induce cancer of C57BL/6 mice and cause muscle atrophy to evaluate whether the composition of the present invention (hereinafter referred to as composition A below) can reduce muscle loss and muscle atrophy of mice due to cancer.

### [Experimental Cell Lines]

Lewis lung carcinoma cells of mice were cultured in RPMI-1640 liquid medium with plus 10% fetal bovine serum (FBS) and appropriate antibiotics (1% penicillin-streptomycin), and placed in an incubator with 5% carbon dioxide and appropriate humidity at 37° C., and the cells were subcultured and the culture medium were replaced 2-3 times a week.

### [Animal Testing]

Orally administer composition A (low dose: 0.5 g and high dose: 1.0 g) or water to mice daily. After dosing for 12 days, mice were injected subcutaneously into the right thigh with LLC cells (1×106 cells). Mice continued to receive composition A or water until the experiment was terminated. Mice in the control group drank water throughout the experiment. Animals were sacrificed on the 32nd day by inhaling carbon dioxide. After inoculated with tumor cells or administration of phosphate buffered physiological saline (PBS), weight and tumor size of the mice were measured once a week.

### [Orally Administration of Composition A Inhibits Tumor Growth and Cachexia Induced by Tumors]

Specifically, organ tumors are characteristic symptom of cancer-induced cachexia in mice.

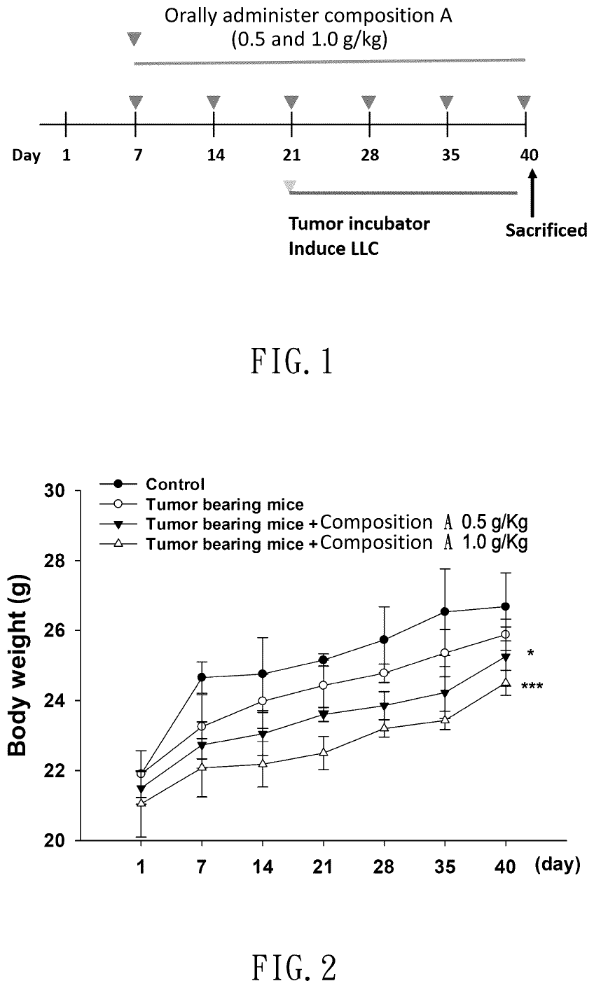

Mice were given oral administration of composition A for 14 days in advance, then inoculated with the aforementioned tumor cells (1×106 LLC cells), and then continued oral administration of composition A until the end of the experiment. FIG. 1 shows the administration course that C57BL/6 mice were inoculated with tumor lung cancer cells and composition A.

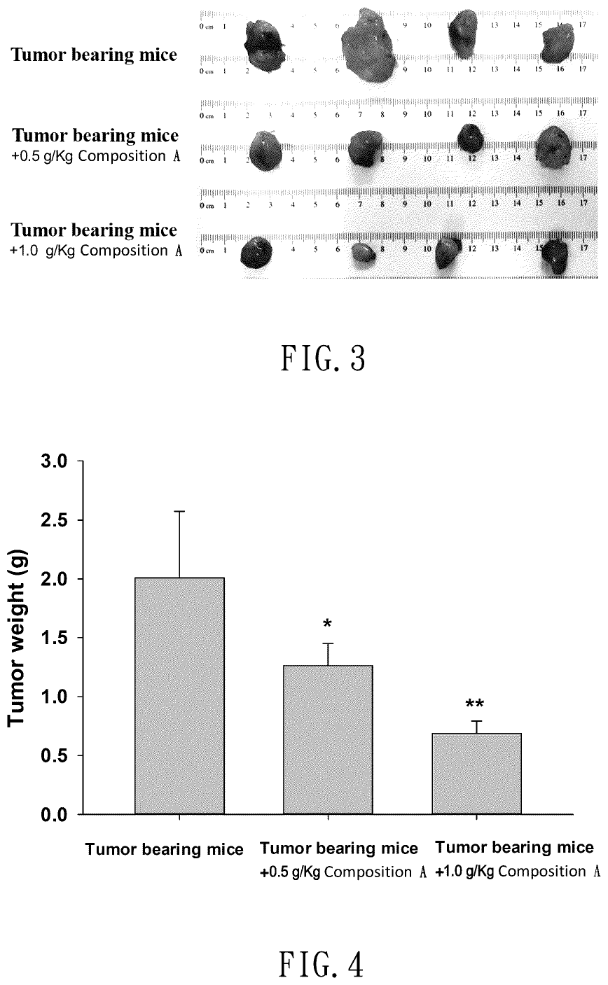

FIG. 2 shows the result of an experiment in which the mice were weighed with the cancer cachexia of mice being excised. The result of an experiment shows that the mice whose was induced in Lewis Lung carcinoma tumors (hereinafter referred to as mice in the experimental group) did not lose weight. Compared with the mice in the control group, the mice in the experimental group took the composition A with a dosage of 0.5 g/Kg (mass of composition A/mass of mouse) orally and the mice in the experimental group took the composition A with a dosage of 1.0 g/Kg were lighter in weight. In tumor prevention, the tumor sizes of mice treated with composition A with a dosage of 0.5 g/Kg and 1.0 g/Kg were significantly reduced compared with those of mice in the control group and mice in the experimental group, as shown in FIGS. 3 and 4. The reason why mice with cancer cachexia induced by Lewis Lung carcinoma tumors did not lose weight may be due to the large size and weight of the tumors in the mice.

### [Composition A Inhibits Tumor-Induced Muscle Atrophy]

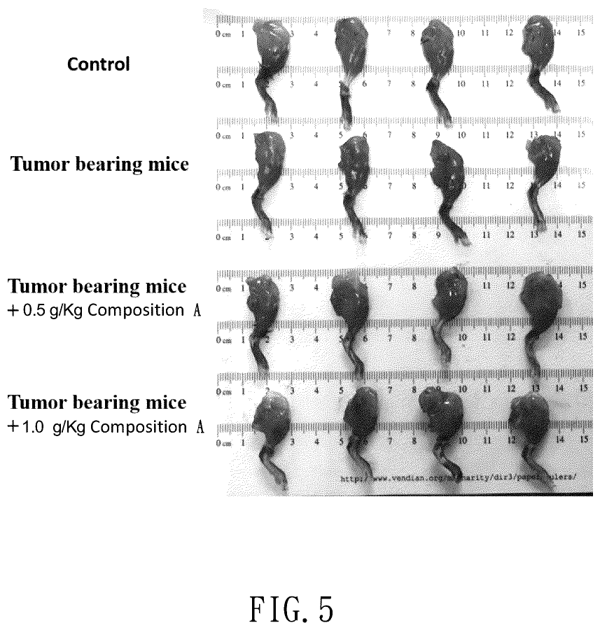

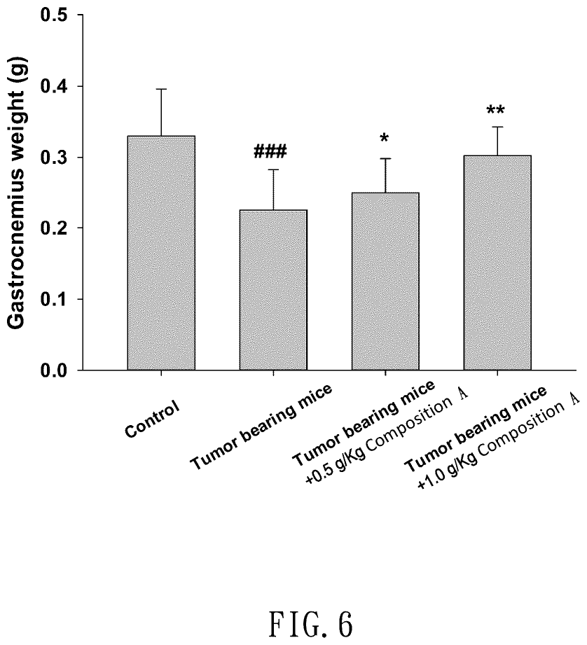

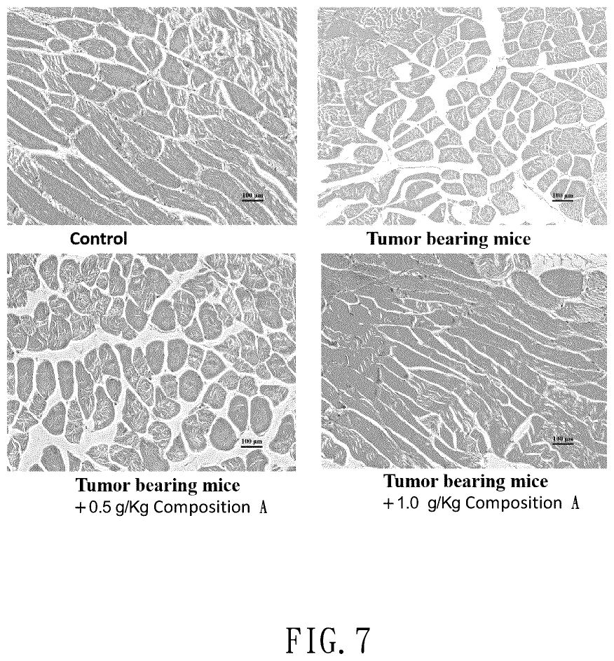

As shown in FIGS. 5 and 6, the experimental results show that the muscle atrophy of the gastrocnemius muscle was visible to the eyes. Compared with the mice in the control group, the weight of the gastrocnemius muscle of the mice in the experimental group was relatively reduced, indicating that Lewis lung carcinoma cells indeed caused muscle atrophy in C57BL/6 mice. Compared with the mice in the experimental group, the gastrocnemius muscle weights of the mice treated with composition A at a dosage of 0.5 g/Kg and 1.0 g/Kg were significantly increased. That is, the gastrocnemius muscle of mice induced by tumors will be reduced due to cancer, while the gastrocnemius muscle of mice treated with composition A will be significantly increased. Furthermore, in the pathological sections of hematoxylin-eosin stain (H&E stain) of gastrocnemius muscle (as shown in FIG. 7), there are cancer cells in the gastrocnemius muscles of mice in the experimental group, and there are no cancer cells in the gastrocnemius muscles of mice treated with composition A at a dosage of 0.5 g/Kg and 1.0 g/Kg.

### [Composition a Inhibits Nitric Oxide]

Respective ones of 100 μL (microliter) serums of mice in the control group, mice in the experimental group, and mice treated with composition A at a dosage of 0.5 g/Kg and 1.0 g/Kg are added in 96-well culture plates, an equal amount (100 μL) of Griess Reagent (mixture of a solution of N-(1-naphthyl) ethylenediamine dihydrochloride (NED) dissolved in water and a solution of sulfanilamide with 1% concentration in phosphoric acid with 5% concentration) into the serums in the 96-well culture plates, respectively, and a light with a wavelength of 540 nm of a microplate spectrometer (Molecular Devices) is used to measure the light absorbance to determine the ability to scavenge free radicals of nitric oxide.

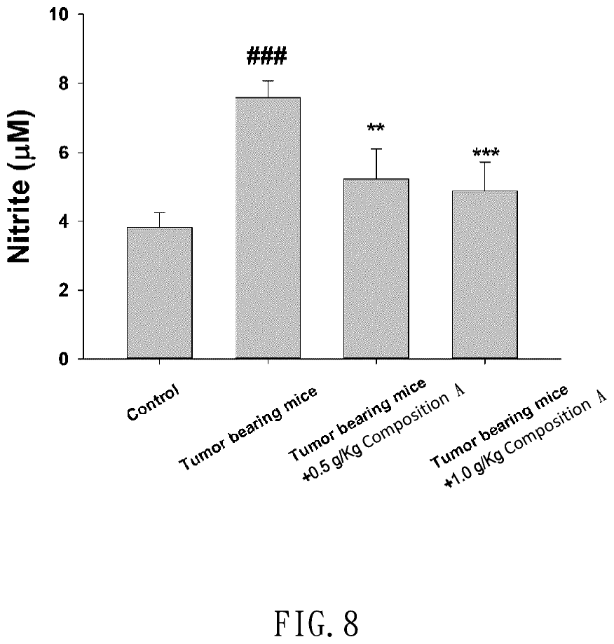

FIG. 8 shows that the nitric oxide in mice treated with composition A at a dosage of 0.5 g/Kg and 1.0 g/Kg was significantly lower than that of mice in the experimental group.

### [Composition A Activates Glutathione]

To measure the amount of glutathione, 720 μL of muscle homogenates of mice in the control group, mice in the experimental group, and mice treated with composition A at a dosage of 0.5 g/Kg and 1.0 g/Kg, in 200 mM of Tris-HCl buffer solution (pH 7.2), were diluted to 1,440 μL, and 160 μL of trichloroacetic acid (TCA) with 5% concentration was added into and mixed with the above mixture thoroughly. The sample was then centrifuged at 10,000 g for 5 minutes at 4° C. The supernatant (330 μL) is placed in a test tube and 660 μL of DTNB (sulfhydryl reagent 5,5′-dithio-bis(2-nitrobenzoic acid)) solution is added into the test tube. Finally, a light with a wavelength of 405 nm of a microplate spectrometer is used to measure the light absorbance.

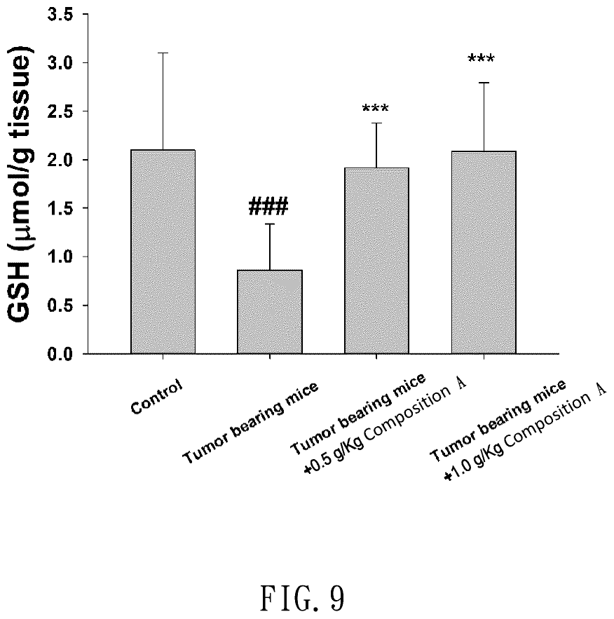

FIG. 9 shows that the glutathione in the muscles of the mice in the experimental group was significantly lower than that of the mice in the control group, while the glutathione in the muscles of the mice treated with composition A at a dosage of 0.5 g/Kg and 1.0 g/Kg was significantly increased.

### [Composition a Inhibits Muscle-Specific E3 Ubiquitin Ligase]

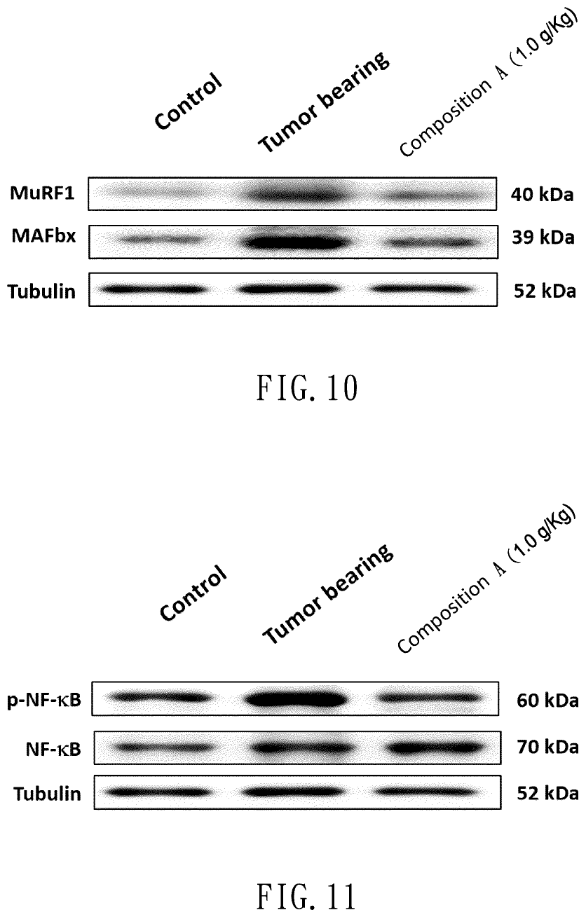

Muscle-specific E3 ubiquitin ligase includes muscle RING-finger 25 protein-1, (MuRF1) and muscle atrophy F-box, (MAFbx). Specifically, muscle RING-finger 25 protein-1 and muscle atrophy F-box are involved in the process of muscle atrophy in cancer cachexia. As shown in FIG. 10, Western blot analysis revealed that the expression level of muscle RING-finger 25 protein-1 and muscle atrophy F-box in the gastrocnemius muscle of the mice in the experimental group was significantly higher than that of the mice in the control group, while the expression level of composition A muscle RING-finger 25 protein-1 and muscle atrophy F-box in the gastrocnemius muscle of the mice treated with composition A at a dosage of 1.0 g/Kg was lower than that of the mice in the experimental group.

### [Composition A Inhibits Inflammatory Protein in Gastrocnemius Muscle]

Dysregulation of the inflammatory protein in the gastrocnemius muscle is associated with cancer, inflammation and autoimmune diseases, septic shock, viral infection and immune development abnormalities. The inflammatory protein is nuclear factor kappa-light-chain-enhancer of activated B cells (NF-κB). As shown in FIG. 11, Western blot analysis revealed that the expression level of p-NF-κB in the muscle tissue of mice in the experimental group was higher than that of mice in the control group. The expression level of p-NF-κB in muscle tissue of mice treated with composition A at a dosage of 1.0 g/Kg was significantly lower than that of the mice in the experimental group. Therefore, from the experimental results, the composition A regulates transduction of ubiquitin-proteasomal proteolysis conducted by p-NF-κB.

The above-mentioned term “inhibition” refers to reduction or improvement. In this embodiment, mice are treated, preferably taken orally, with composition A at a dosage of 0.5 g/Kg and 1.0 g/Kg, but not limited thereto.

In sum, the composition of the present invention is capable of inhibiting tumor growth and cachexia induced by tumors, activating glutathione, inhibiting muscle-specific ubiquitin ligase, inhibiting inflammatory protein in gastrocnemius muscle, inhibiting nitric oxide, and inhibiting tumor-induced muscle atrophy.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A composition including 70.00-99.00 weight percentage (wt %) of phytopeptide, 0.10-20.00 wt % of branched-chain amino acid, 0.10-20.00 wt % of whey protein concentrate, 0.10-20.00 wt % of creatine, and 0.10-20.00 wt % of chromium yeast.

2. The composition of claim 1, wherein the composition includes 85.90-95.90 wt % of phytopeptide, 0.25-1.25 wt % of branched-chain amino acid, 1.85-5.85 wt % of whey protein concentrate, 1.00-5.00 wt % of creatine, and 0.50-2.00 wt % of chromium yeast.

3. The composition of claim 1, wherein the weight of the phytopeptide is from 2834.70 mg to 3164.70 mg, the weight of the branched-chain amino acid is from 8.25 mg to 41.25 mg, the weight of the whey protein concentrate is from 61.40 mg to 193.05 mg, the weight of the creatine is from 33.00 mg to 165.00 mg, and the weight of the chromium yeast is from 16.50 mg to 66.00 mg.

4. The composition of claim 1, wherein the phytopeptide is ipomoea batatas phytopeptide.

5. A use of the composition of claim 1 for manufacturing food for prevention of sarcopenia.

6. The use of the composition of claim 5 for manufacturing food for prevention of sarcopenia, wherein the food is capable of inhibiting tumor growth and cachexia induced by tumors.

7. The use of the composition of claim 5 for manufacturing food for prevention of sarcopenia, wherein the food is capable of activating glutathione.

8. The use of the composition of claim 5 for manufacturing food for prevention of sarcopenia, wherein the food is capable of inhibiting muscle-specific ubiquitin ligase.

9. The use of the composition of claim 5 for manufacturing food for prevention of sarcopenia, wherein the food is capable of inhibiting inflammatory protein in gastrocnemius muscle.

10. The use of the composition of claim 5 for manufacturing food for prevention of sarcopenia, wherein the food is capable of inhibiting tumor growth and cachexia induced by tumors, activating glutathione, inhibiting muscle-specific ubiquitin ligase, inhibiting inflammatory protein in gastrocnemius muscle, inhibiting nitric oxide, and inhibiting tumor-induced muscle atrophy; the muscle-specific ubiquitin ligase includes at least one of muscle RING-finger 25 protein-1 (MuRF1) and muscle atrophy F-box (MAFbx); the inflammatory protein is nuclear factor kappa-light-chain-enhancer of activated B cells (NF-κB).
